(19) United States
(12) Patent Application Publication
(10) Pub. No.: US 2016/0101081 A1
(43) Pub. Date: Apr. 14, 2016

(54) COMPOSITIONS AND METHODS FOR ENHANCING MOBILITY OR ACTIVITY OR TREATING FRAILTY

(71) Applicant: NESTEC SA, Vevey (CH)

(72) Inventors: Yuanlong Pan, Chesterfield, MO (US); Janet R. Jackson, Columbia, IL (US)

(21) Appl. No.: 14/879,600

(22) Filed: Oct. 9, 2015

Related U.S. Application Data
(60) Provisional application No. 62/062,283, filed on Oct. 10, 2014.

(51) Int. Cl. A61K 31/23; A23K 1/16; A61K 45/06

(52) U.S. Cl. CPC A61K 31/23; A61K 45/06; A23K 1/164; A23K 1/1643; A23K 1/1631; A23K 1/1603

(57) ABSTRACT

The present invention provides a method for increasing mobility or activity in an animal or treating or preventing frailty in an animal, comprising identifying an animal with decreased mobility or activity or having frailty and administering a composition comprising one or more medium chain triglycerides (MCTs) to the animal in a therapeutically amount effective for increasing mobility or activity in the animal or treating frailty in the animal. Additionally, a food composition can comprise about 15% to about 50% protein, about 5% to about 40% fat, about 5% to about 50% carbohydrate, and about 1% to about 40% of one or more medium chain triglycerides (MCTs); wherein the composition has a moisture content of about 5% to about 20% and the MCTs are present in a therapeutically amount effective for increasing mobility or activity in the animal or treating frailty in the animal.

### CROSS REFERENCE TO RELATED APPLICATIONS

This application claims priority to U.S. Provisional Application Ser. No. 62/062,283 filed Oct. 10, 2014, the disclosure of which is incorporated in its entirety herein by this reference.

### BACKGROUND OF THE INVENTION

1. Field of the Invention

The invention relates generally to compositions and methods for increasing mobility and activity in animals and treating or preventing frailty in animals, and particularly to compositions comprising medium chain triglycerides and their use for increasing mobility and activity in animals, and treating or preventing frailty in animals.

2. Description of Related Art

According to the Human Society, in 2012, 62 percent of American households included at least one pet, with a total of 179 million owned pets in the U.S. This corresponds to approximately 83 million dogs and 96 million cats.

Elderly pets often become frail in their last few years of life, often having joint stiffness, energy loss, and/or reduced activity levels. For example, the connective tissues of animals are constantly subject to stresses and strains from mechanical forces that can result in afflictions, such as arthritis (both rheumatoid and osteoarthritis), joint inflammation and stiffness. This is particularly true as animals age.

Quality of life has become an ever important topic as the advancement of veterinary science has led to increased life spans for these animals. Accordingly, experts in the veterinary field have increasingly observed and reported problems which typically occur as these animals grow older (see Journal of the American Veterinary Medical Association, Vol. 218, No. 11, Pages 1787-1791 (2001), and Journal of the American Veterinary Medical Association, Vol. 218, No. 11, Pages 1792-1795 (2001)). These problems include difficulty breathing, forced or labored breathing, leg trembling, difficulty walking, decreased reaction times, decreased excretion, failure of one or more of the five senses, loss of directional sense, inactivity, and the like.

Further, frailty is a major health problem affecting older adults, and it was estimated that pre-frail and frail adults account for almost 50% of people aged 60 and older. (The Journal of Nutrition, Health & Aging 16:283-284, 2012).

As such, research and development efforts continue in the area of animal research to develop effective nutritional intervention to increase activity and mobility, and prevent and treat frailty in animals.

### SUMMARY OF THE INVENTION

It is, therefore, an object of the present invention to provide compositions and methods useful for increasing mobility and/or activity and treating and/or preventing frailty in an animal.

In one embodiment, a method for increasing mobility or activity in an animal or treating frailty in an animal can comprise identifying an animal with decreased mobility or activity or having frailty and administering a composition comprising one or more medium chain triglycerides (MCTs) to the animal in a therapeutically amount effective for increasing mobility or activity in the animal or treating frailty in the animal.

Additionally, in another embodiment, a pet food composition can comprise about 15% to about 50% protein, about 5% to about 40% fat, about 5% to about 50% carbohydrate, and about 1% to about 40% one or more medium chain triglycerides (MCTs). The composition can have a moisture content of about 5% to about 20% and the MCTs can be present in a therapeutically amount effective for increasing mobility or activity in the animal or treating frailty in the animal.

Other and further objects, features, and advantages of the present invention will be readily apparent to those skilled in the art.

### DETAILED DESCRIPTION OF THE INVENTION

### Definitions

The following abbreviations may be used herein: MCTs, medium chain triglycerides; ALA, alpha-linolenic acid; DHA, docosahexaenoic acid; DPA, docosapentaenoic acid; EPA, eicosapentaenoic acid; LA, linoleic acid; LCPUFA, long chain polyunsaturated fatty acids (as used herein LCPUFA refers to one or more such fatty acids); NO, nitric oxide; NORC, nitric oxide releasing compound or compounds; and L-Arg, L-arginine.

The term “animal” means any animal that could benefit from one or more of the methods or compositions of the present invention including those for increasing mobility and/or activity. Generally, the animal can be a human, avian, bovine, canine, equine, feline, hicrine, lupine, murine, ovine, and porcine animal. In one aspect, the animal can be a companion animal. A “companion animal” means any domesticated animal, and includes, without limitation, cats, dogs, rabbits, guinea pigs, ferrets, hamsters, mice, gerbils, horses, cows, goats, sheep, donkeys, pigs, and the like. In one specific aspect, the animal or companion animal can be a dog or cat.

The term “increasing mobility and/or activity” or “increasing mobility or activity” means one or more of increasing mobility and activity in an animal. As such, both phrases can include one or both effects.

The term “treating frailty” means that any condition associated with frailty of an animal is lessened or minimized, including without limitation, weakness, slowing, decreased energy, lower activity, and unintended weight loss.

The term “therapeutically effective amount” means an amount of a compound of the invention that (i) treats or prevents or minimizes the particular disease, condition, or disorder, e.g. decreased mobility or activity (ii) attenuates, ameliorates, or eliminates one or more symptoms of the particular disease, condition, or disorder, e.g. lack of movement, lethargic behavior, or (iii) prevents or delays the onset of one or more symptoms of the particular disease, condition, or disorder described herein.

The term “medium chain triglycerides” or “MCTs” means any glycerol molecule ester-linked to three fatty acid molecules, each fatty acid molecule having 6-12 carbons. MCTs may be represented by the following general formula (Formula I):

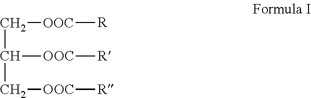

where R, R′ and R″ are fatty acids having 6-12 carbons in the carbon backbone esterified to a glycerol backbone. The lipids of the invention may be prepared by any process known in the art, such as direct esterification, rearrangement, fractionation, transesterification, or the like. For example, the lipids may be prepared by the rearrangement of a vegetable oil such as coconut oil. The length and distribution of the chain length may vary depending on the source oil. For example, MCTs containing 1-10% C6, 30-60% C8, 30-60% C10, 1-10% C12 are commonly derived from palm and coconut oils. MCTs containing greater than about 95% C8 at R, R′ and R″ can be made by semi-synthetic esterification of octanoic acid to glycerin. Mixtures comprising MCTs with about 50% total C8 and/or about 50% total C10 can also be used. Commercial sources for the foregoing MCT compositions are available and known to the skilled artisan. Such MCTs behave similarly and are encompassed within the term MCTs as used herein.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. “Healthy aging animals” are those with no known diseases, particularly diseases relating to loss of cognitive impairment such as might confound the results. In studies using healthy aging animals, cohort animals are generally also healthy aging animals, although other healthy animals with suitable mobility or activity functioning may be suitable for use as comparative specimens. If animals with specific disease diagnoses, or cognitive, motor, or behavioral limitations are used, then the cohort animals should include animals that are similarly diagnosed, or which present with similar indicia of the disease or cognitive, motor, or behavioral limitation.

The term “food” or “food product” or “food composition” means a product or composition that is intended for ingestion by an animal, including a human, and provides nutrition to the animal.

As used herein, a “food product formulated for human consumption” is any composition specifically intended for ingestion by a human being. The term “pet food” or “pet food composition” means a composition intended for consumption by animals, and in one aspect, by companion animals.

The term “complete and balanced” or “nutritionally balanced” when referring to a food composition means a food composition that contains all known required nutrients in appropriate amounts and proportions based on recommendations of recognized authorities in the field of animal nutrition, and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO). The term includes any food, feed, snack, food supplement, treat, meal substitute, or meal replacement, whether intended for a human or another animal. Animal food includes food or feed intended for any domesticated or wild species. In some embodiments, a food for an animal represents a complete and balanced food composition, e.g., a pelleted, extruded, or dry food. Examples of such animal foods include extruded pet foods, such as foods for dogs and cats.

“RDA” or “recommended daily allowance” means levels set by the U.S.D.A. as last published during the year of 2013.

The term “dietary supplement” means a product that is intended to be ingested in addition to the normal animal diet. Dietary supplements may be in any form, e.g., solid, liquid, gel, tablets, capsules, powder, and the like. In one embodiment, they can be provided in convenient dosage forms. In some embodiments, they can be provided in bulk consumer packages such as bulk powders, liquids, gels, or oils. In other embodiments, supplements can be provided in bulk quantities to be included in other food items such as snacks, treats, supplement bars, beverages and the like.

The term “long-term administration” means periods of repeated administration or consumption in excess of one month. Periods of longer than two, three, or four months can be used for certain embodiments. Also, extended periods can be used that include longer than 5, 6, 7, 8, 9, or 10 months. Periods in excess of 11 months or 1 year can also be used. Longer term use extending over 1, 2, 3, or more years can also be used. For certain aging animals, the animal can continue consuming on a regular basis for the remainder of its life. Sometimes this can be referred to as consumption for “extended” periods.

The term “regular basis” means at least monthly dosing with the compositions or consumption of the compositions, and in one aspect, weekly dosing. More frequent dosing or consumption, such as twice or three times weekly, can also be performed in certain embodiments. Still, in other aspects, regimens that comprise at least once daily consumption can be employed. The skilled artisan will appreciate that the blood level of a compound or certain metabolites of that compound or which result after the consumption of that compound, may be a useful tool for assessing or determining dosing frequency. For example, for determining dosage or dosage frequency for compositions comprising MCTs, the blood concentration of ketone bodies, or a specific ketone body may provide useful information. A frequency, regardless of whether expressly exemplified herein, that allows maintenance of a desired blood level of the measured compound within acceptable ranges is useful herein. The skilled artisan will appreciate that dosing frequency will be a function of the composition that is being consumed or administered, and some compositions may require more or less frequent administration to maintain a desired blood level of the measured compound (e.g., a ketone body).

The term “oral administration” or “orally administering” means that the animal ingests, or a human is directed to feed, or does feed, the animal one or more of the compositions described herein. Wherein a human is directed to feed the composition, such direction may be that which instructs and/or informs the human that use of the composition may and/or will provide the referenced benefit, for example, increasing mobility or activity, or preventing, reducing, or delaying a decline in such foregoing functions or qualities. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, website, or other computer-related media), and/or packaging associated with the composition (e.g., a label present on a container holding the composition), or a combination thereof (e.g., label or package insert with directions to access a website for more information).

The term “in conjunction” means that a composition comprising MCTs, a food composition, or other compound or composition of the present invention are administered to an animal (1) together in a food composition or (2) separately at the same or different frequency using the same or different administration routes at about the same time or periodically. “Periodically” means that the agent is administered on a dosage schedule acceptable for a specific agent and that the food is fed to an animal routinely as appropriate for the particular animal. “About the same time” generally means that the food and agent are administered at the same time or within about 72 hours of each other. “In conjunction” specifically includes administration schemes wherein a composition or drug is administered for a prescribed period and the compositions comprising MCTs are administered indefinitely.

The term “individual” when referring to an animal means an individual animal of any species or kind.

The term “microorganism” encompasses at least bacteria, molds and other fungi, and yeasts. Probiotics are beneficial microorganisms that can survive or even multiply and thrive in the gastrointestinal tract of an animal. Probiotics can contribute to the overall health of an animal generally and particularly to the gastrointestinal health of the animal.

The term “single package” means that the components of a kit are physically associated, in or with one or more containers, and considered a unit for manufacture, distribution, sale, or use. Containers include, but are not limited to, bags, boxes or cartons, bottles, packages of any type or design or material, over-wrap, shrink-wrap, affixed components (e.g., stapled, adhered, or the like), or combinations of any of the foregoing. For example, a single package kit may provide containers of individual compositions and/or food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain, for example, instructions on how to use the kit, or safety or technical information about one or more components of a kit. Examples of information that can be provided as part of a virtual kit include instructions for use; safety information such as material safety data sheets; poison control information; information on potential adverse reactions; clinical study results; dietary information such as food composition or caloric composition; general information on behavioral or motor function; diseases that effect behavioral or motor function; treating behavioral or motor function; or general information on treatment or preservation of behavioral or motor function; self-help relating to behavioral or motor function; caregiver information for those caring for animals with behavioral or motor function challenges; and use, benefits, and potential side-effects or counter-indications for the compositions or drugs.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used throughout, ranges are used herein in shorthand, so as to avoid having to set out at length and describe each and every value within the range. Any appropriate value within the range can be selected, where appropriate, as the upper value, lower value, or the terminus of the range.

The term “about” means plus or minus 20%; in one aspect, plus or minus 10%; in another aspect, plus or minus 5%; and in one specific aspect, plus or minus 2%.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a puppy”, “a method”, or “a food” includes a plurality of such “puppies”, “methods”, or “foods”. Reference herein, for example to “an antioxidant” includes a plurality of such antioxidants, whereas reference to “pieces” includes a single piece. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

As used herein, embodiments, aspects, and examples using “comprising” language or other open-ended language can be substituted with “consisting essentially of” and “consisting of” embodiments.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

In one aspect, the invention provides methods and compositions suitable for increasing mobility and/or activity and preventing and/or treating frailty in an animal. The present inventors have discovered that compositions comprising MCTs can be effective for increasing mobility and/or activity and preventing or treating frailty in animals. As such, a method for increasing mobility or activity or preventing or treating frailty in an animal can comprise identifying an animal with decreased mobility or activity or having frailty, or being at a risk from frailty; and administering a composition comprising one or more medium chain triglycerides (MCTs) to the animal in a therapeutically amount effective for increasing mobility or activity and preventing or treating frailty in the animal. Further, a pet food composition suitable for increasing mobility or activity in an animal can comprise about 15% to about 50% protein, about 5% to about 40% fat, about 5% to about 50% carbohydrate, and about 1% to about 40% one or more medium chain triglycerides (MCTs). The composition can have a moisture content of about 5% to about 20% and the MCTs can be present in a therapeutically amount effective for increasing mobility or activity and prevent or treating frailty in the animal.

The present methods and compositions can be useful for, adult or senior animals. In one embodiment, the animal can be a companion animal. In one aspect, the animal can be a cat or dog. Additionally, in another embodiment, the animal can be an aging animal.

Generally, the present invention can use any type of MCT suitable for administration to an animal. In one embodiment, the MCTs can have the structure of Formula I:

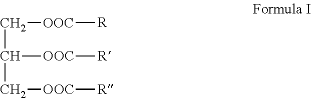

where the R, R′ and R″ esterified to the glycerol backbone are each independent fatty acids having 6-12 carbons. In one aspect, greater than about 95% of the R, R′ and R″ can be 8 carbonsin length. In another aspect, the remaining R, R′ and R″ can be 6-carbon or 10-carbon fatty acids. In other embodiments, greater than at least or about 30, 40, or 50% of R, R′ and R″ can be C8, and/or greater than at least or about 30, 40, or 50% of R, R′ and R″ can be C10. In one embodiment, about 50% of the R, R′ and R″ can be C8 and about 50% of R, R′ and R″ can be C10. MCTs can be obtained from any suitable source, synthetic or natural. Examples of natural sources of MCT include plant sources such as coconuts and coconut oil, palm kernels and palm kernel oils, and animal sources such as milk from any of a variety of species.

Typically, the MCTs can be administered as part of a pet food composition. Additionally, the MCTs can be administered to the animal by adding the MCTs to an existing pet food composition prior to feeding the existing pet food composition to the animal. In one aspect, the pet food composition can be formulated as a complete and nutritionally balanced pet food. In another aspect, the pet food composition can be formulated as a dry extruded food, as a treat, or as a semi-moisture pet food composition. In certain embodiments, the compositions further comprise one or more B vitamins, one or more antioxidants, or combinations thereof.

In one embodiment, the methods and compositions discussed herein can include the use of a long chain polyunsaturated fatty acid (LCPUFA). The LCPUFA can be any LCPUFA suitable for administration to an animal. LCPUFAs can be obtained from any suitable source, synthetic or natural. Sources of LCPUFA include natural sources of such fatty acids such as, without limitation, primrose; dark green vegetables such as spinach; algae and blue-green algae such as spirulina; plant seeds and oils from plants such as flax, canola, soybean, walnut, pumpkin, safflower, sesame, wheat germ, sunflower, corn, and hemp; and fish such as salmon, tuna, mackerel, herring, sea bass, striped bass, halibut, catfish, sardines, shark, shrimp, and clams; and the extracted oils of any one or more of the foregoing. The LCPUFA may also be synthetic, and as such may be produced according to any means suitable in the art, from any suitable starting material. In certain embodiments, the LCPUFA comprise one or more (n-3) fatty acids such as ALA, EPA, DPA and DHA. The presently disclosed LCPUFAs may comprise a blend of any one or more LCPUFA from any one or more sources, such as those exemplified above, whether natural or synthetic.

In one embodiment, the methods and compositions discussed herein can include the use of a nitric oxide releasing compound (NORC). The NORCs can be any NORC suitable for administration to an animal. NORCs can be obtained from any suitable source, synthetic or natural. In various embodiments, the NORC comprises arginine. Sources of arginine include, without limitation, animal and plant proteins. Examples of plants considered rich in arginine content and suitable for use herein include, but are not limited to, legumes such as soy, lupins, and carob; grains such as wheat and rice; and fruits such as grapes. Seeds and nuts of plants such as cacao and peanut are also considered rich in arginine content and are therefore useful herein. Some examples of suitable animal proteins considered rich in arginine content are poultry and fish products. In certain embodiments, the NORC comprise one or more of L-Arg and nitric oxide-releasing derivatives thereof. The NORC can also be synthetically produced, according to any suitable means in the art. As with LCPUFA, the NORC content of any composition disclosed herein can include a blend of any natural or synthetic NORC. Both LCPUFA and NORC, whether natural or synthetic, can be obtained directly or provided by a commercial source.

In one aspect, the methods and compositions can include one or more B vitamins, one or more antioxidants, or combinations thereof. The B vitamins can be any B vitamin suitable for administration to an animal. B vitamins include vitamins B1 (thiamine), B2 (riboflavin), B3 (aka P or PP) (niacin, including nicotinic acid and/or nicotinamide), B5 (pantothenic acid), B6 (pyridoxine), B7 (aka H) (biotin), B8 (myo-inositol), B9 (aka M or B-c) (folic acid), B12 (cobalamin), or salts, conjugates, or derivatives thereof recognized of found to have B vitamin activity. Combinations of any of the foregoing can also be useful herein and are sometimes referred to herein as “mixtures” of B vitamins. Since the vitamin requirements vary for different species, not all of the listed compounds are deemed vitamins for all species. For example, since it is known that myo-inositol can be synthesized by humans, it is no longer deemed a vitamin, as it is not required for adequate human nutrition. The antioxidants can be any antioxidant suitable for administration to an animal. Antioxidants are well known in the art, particularly the art of food technology and food formulation. Natural antioxidant compounds include vitamins (such as A, C and E, and derivative, conjugates, or analogs thereof), as well as plant extracts, including extracts from fruit, vegetables, herbs, seeds, and other types and/or parts of plants. Compounds such as α-lipoic acid, chlorophyll and derivatives thereof, glutathione, ubiquinols (e.g., coenzyme Q10), carotenoids (e.g., lycopene), flavonoids, phenolic acids and polyphenols, and pycnogenol are known to be excellent antioxidants. Some examples of plant sources of antioxidants include those from fruits such as berries (cherry, blackberry, strawberry, raspberry, crowberry, blueberry, bilberry/wild blueberry, black currant), pomegranate, grape, orange, plum, pineapple, kiwi fruit, and grapefruit; those from vegetables including kale, chili pepper, red cabbage, peppers, parsley, artichoke, Brussels sprouts, spinach, lemon, ginger, garlic, and red beets; those from dry fruits like apricots, prunes, and dates; from legumes including broad beans, pinto beans, and soybeans. Also nuts and seeds such as pecans, walnuts, hazelnuts, ground nut, and sunflower seeds; cereals such as barley, millet, oats, and corn. Many natural antioxidants are also available from a wide variety of spices including cloves, cinnamon, rosemary, and oregano. Less widely known sources of antioxidants include Ginkgo biloba, and tropical plants such as uyaku, and carica papaya. Antioxidant properties of various teas and green tea, as well as fermented products such as red wine, have become of great interest in recent years and such would be suitable for use herein. Selenium is an excellent oxygen scavenger and works well, especially with vitamin or related tocopherol compounds. Synthetic dietary antioxidants include butylated hydroxyanisole (BHA) and butylated hydroxytoluene (BHT) which are commonly used in food products. Any of the foregoing, alone or in combination, can be suited for use herein, as can be combinations of natural and synthetic antioxidants

The MCTs, and if present, LCPUFA, NORC, B vitamins and antioxidants, can be present in an amount effective for activity and mobility. Generally, MCTs can be present in an amount from about 1% to about 40%. In one aspect, the MCTs can be present in an amount from about 2.5% to about 20%, from about 5 to about 15%, from about 5% to 10%, or even from about 5% to about 8%.

If present, the compositions can comprise from about 0.1% to about 50% LCPUFA and from about 0.1% to about 20% NORC. In various embodiments, the compositions comprise from about 1 to about 30% LCPUFA, and in one aspect, from about 1 to about 15% LCPUFA; from about 1 to about 15% NORC, and in one aspect, from about 1 to about 10% NORC. Additionally, B vitamins and antioxidants can be present in any acceptable amount including recommended daily amounts. In one embodiment, the composition comprises from about 0.1 to 40 times the recommended daily requirement (RDA) of B vitamins and from about 0.01 to 25 times the RDA of antioxidants. In various embodiments, the B vitamins comprise from about 4 to 20 times the RDA, and in one aspect, from about 1 to 10 times the RDA, and antioxidants comprise from about 0.01 to 15 times the RDA, and in one aspect, from about 0.01 to 5 times the RDA, or even from about 0.01 to 2 times the RDA. In one embodiment, the compositions comprise LCPUFA, NORC, and MCTs, and a mixture of one or more B vitamins and one or more antioxidants in such amounts.

The compositions may further comprise substances such as minerals, other vitamins, salts, functional additives including, for example, palatants, colorants, emulsifiers, antimicrobial or other preservatives. Minerals that may be useful in such compositions include, for example, calcium, phosphorous, potassium, sodium, iron, chloride, boron, copper, zinc, magnesium, manganese, iodine, selenium and the like. Examples of additional vitamins useful herein include such fat soluble vitamins as A, D, E, and K. Inulin, amino acids, enzymes, coenzymes, and the like may be useful to include in various embodiments.

In one embodiment, the compositions can be food compositions, including human and pet food compositions. Such compositions include foods intended to supply the necessary dietary requirements for an animal, animal treats (e.g., biscuits), or dietary supplements. The compositions may be a dry composition (e.g., kibble), semi-moist composition, wet composition, or any mixture thereof. In another embodiment, the composition can be a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of MCTs, and optionally, LCPUFA, NORC, B vitamins and antioxidants. This permits the supplement to be administered to the animal in small amounts, or in the alternative, can be diluted before administration to an animal. The dietary supplement may require admixing, or can be admixed with water or other diluent prior to administration to the animal. In another embodiment, the MCTs can be pre-blended with the other components to provide the beneficial amounts needed. In yet other embodiments, the MCTs can be used to coat a food, snack, pet food composition, or pet treat. In one embodiment, the MCTs can be added to the composition just prior to offering it to the animal, e.g., using a sprinkled powder or a mix.

The compositions can optionally comprise one or more supplementary substances that promote or sustain general health. Such substances may be associated with improved mental health or enhanced cognitive function or may be substances that inhibit, delay, or decrease loss of cognitive function, e.g., herbs or plants that enhance cognitive function.

In various embodiments, pet food or pet treat compositions comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The compositions may further comprise from about 5% to about 40% fat. The compositions may further comprise a source of carbohydrate. The compositions may comprise from about 5% to about 50% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the composition ranges from less than 1% to about 15%, and in one aspect, from about 5% to about 10%.

The moisture content can vary depending on the nature of the composition. In one embodiment, the composition can be a complete and nutritionally balanced pet food. In this embodiment, the pet food may be a “wet food”, “dry food”, or food of intermediate moisture content. “Wet food” describes pet food typically sold in cans or foil bags, and has a moisture content typically in the range of about 70% to about 90%. “Dry food” describes pet food of a similar composition to wet food, but contains a limited moisture content, typically in the range of about 5% to about 15% or 20%, and therefore can be presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also dry food compositions can include extruded food products, such as pet foods, or snack foods for either humans or companion animals.

The compositions may also comprise one or more fiber sources. The term “fiber” includes all sources of “bulk” in the food whether digestible or indigestible, soluble or insoluble, fermentable or nonfermentable. Fibers include plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present invention to the immune system of an animal.

In other embodiments, the compositions further comprise prebiotics or probiotics. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. Prebiotics include fructooligosaccharides (FOS), xylooligosaccharides (XOS), galactooligosaccharides (GOS), and mannooligosaccharides (typically for non-human foods such as petfoods). The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophillus, Bifidobacteria, Lactococcus, and Pediococcus. Beneficial species include Enterococcus and Saccharomyces species. The amount of probiotics and prebiotics to be administered to the animal can be determined by the skilled artisan based upon the type and nature of the prebiotic and probiotic and the type and nature of the animal, e.g., the age, weight, general health, sex, extent of microbial depletion, presence of harmful bacteria, and diet of the animal. Generally, probiotics can be administered to the animal in amounts of from about one to about twenty billion colony forming units (CFUs) per day for the healthy maintenance of intestinal microflora, in one aspect, from about 5 billion to about 10 billion live bacteria per day. Generally, prebiotics can be administered in amounts sufficient to positively stimulate the healthy microflora in the gut and cause these “good” bacteria to reproduce. Typical amounts can be from about one to about 10 grams per serving or from about 5% to about 40% of the recommended daily dietary fiber for an animal. The probiotics and prebiotics can be made part of the composition by any suitable means. Generally, the agents can be mixed with the composition or applied to the surface of the composition, e.g., by sprinkling or spraying. When the agents are part of a kit, the agents can be admixed with other materials or in their own package.

The compositions and dietary supplements may be specially formulated for the intended recipients or consumers, such as for adult animals or for older or young animals. For example, a composition adapted for puppies or kittens or adapted for active, pregnant, lactating, or aging animals can be prepared. In general, specialized compositions will comprise energy and nutritional requirements appropriate for animals at different stages of development or age.

Certain aspects of the invention can be used in combination with a complete and balanced food. According to certain embodiments provided herein, the compositions comprising the MCTs can be used with a high-quality commercial food. As used herein, “high-quality commercial food” refers to a diet manufactured to produce the digestibility of the key nutrients of 80% or more, as set forth in, for example, the recommendations of the National Research Council above for dogs, or in the guidelines set forth by the Association of American Feed Control Officials. Similar high nutrient standards would be used for other animals.

In one embodiment, the food compositions comprise any of a variety of ingredients or combinations thereof selected for their contributions to the overall composition. Thus a skilled food technologist may choose from among natural (e.g., plant or plant-derived, animal or animal-derived, and microbial or microbially-derived), and synthetic ingredients or components. In particular embodiments, the ingredients may include any of the cereal grains and/or fractions or components thereof, meat and meat by-products, fish, shellfish, or other seafood, other animal products or by-products, eggs from any source, vitamins, minerals, salts, sweeteners, fiber, flavoring or other palatants, coloring, and functional ingredients such as emulsifiers, stabilizers, softeners, functional coatings, and the like. Cereals useful in the invention include all plants recognized as “cereal” crops, whether currently used in commercial agriculture or merely known practically or botanically as being a “cereal”. For example, “cereals” includes corn, wheat, rice, barley, sorghum, millet, oats, rye, triticale, buckwheat, fonio, and quinoa. The skilled artisan will appreciate that in a given food composition, the use of one or more such cereal products can be common. Meats useful in the invention include products from any animal, including muscle tissue such as chicken or other poultry, lamb, sheep, veal, beef, or pork. Other animal products and by-products useful in the invention include dairy products or by-products derived from the milk of any species. Other important components or ingredients include fats and the skilled artisan will appreciate that many sources of vegetable, animal, or microbial fats are available for formulating food compositions. In one embodiment, the source of fat can be a plant fat such as corn, soy, or canola oil, including those that are readily available. In another embodiment, an animal fat, such as tallow, can be useful for providing calories from fat, as well as enticing flavor to meat-eating animals. Of course, combinations of any of the foregoing ingredients, such as fats, are known in the art and useful for optimizing the food compositions based on functional properties as well as price and availability.

The skilled artisan will also appreciate that in formulating the food compositions of the invention, the formulation may vary slightly, so as to allow consideration by the formulator of the price and/or availability of certain ingredients in the compositions, as well as the batch-to-batch variation in the analysis of certain ingredients. Thus a given food composition or formulation may vary slightly from batch to batch, plant to plant, or even season to season depending on such factors. Notwithstanding such variation in specific ingredients selected for manufacturing a particular batch of a food composition, the overall composition (for example, analysis of protein, carbohydrate, fat, fiber, or other component) may be held constant or at least substantially constant, for example, in accordance with a label claim, such as a claim or guarantee of a minimum or maximum percent of a particular component.

For pet foods and food products formulated for human consumption, the amount of MCTs as a percentage of the composition can be in the range of about 1% to about 40% of the composition, although a lesser or greater percentage can be supplied. In various embodiments, the amount can be about 1%, 1.5%, 2%, 2.5%, 3%, 3.5%, 4%, 4.5%, 5.0%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5% 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, or more, of the composition. Dietary supplements may be formulated to contain several fold higher concentrations of MCTs, to be amenable for administration to an animal in the form of a tablet, capsule, liquid concentrate, or other similar dosage form, or to be diluted before administration, such as by dilution in water, spraying or sprinkling onto a pet food, and other similar modes of administration. For a dietary supplement, MCTs alone may be administered directly to the animal or applied directly to the animal's regular food. Dietary supplement formulations in various embodiments contain about 30% to about 100% MCTs, although lesser amounts may also be used.

For pet foods and food products formulated for human consumption, the amount of LCPUFA as a percentage of the composition can be in the range of about 0.1% to about 13% of the composition, although a greater percentage can be supplied. In various embodiments, the amount of LCPUFA can be about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more, e.g., 6%, 7%, 8%, 9%, 10%, 11%, 12%, 13%, 14%, 15%, 16%, 17%, 18%, 19%, 20% or more, of the composition. Up to 30, 40, or LCPUFA may be used in certain embodiments.

For pet foods and food products formulated for human consumption, the amount of NORC as a percentage of the composition can be in the range of about 0.1% to about 12% of the composition, although a greater percentage can be supplied. In various embodiments, the amount of NORC can be about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more, e.g., 6%, 7%, 8%, 9%, 10%, 11%, 12% or more, up to about 15% or even 20% of the composition.

In one embodiment, the food compositions comprise a macronutrient composition suitable for the type of food being designed. In one embodiment, the food composition can have about 20 to 32% protein, about 30 to 50% carbohydrate, about 5% to 20% fat, and about 15% to 25% moisture. In another embodiment, the food composition can be a pet food composition such as a premium or super-premium pet food composition. In one embodiment, the pet food can be formulated for canines and has a protein content of about 20-30%, in one aspect, about 24-28%, or even about 25-27%. In one embodiment, the protein content of a dog food composition can be about 26% by weight. In another embodiment, the formulation can be for felines and can have a protein content of about 35-45%, in one aspect, about 37-42%, or even about 39-41%. In one embodiment, the protein content of a cat food composition can be about 40%.

In another aspect, the compositions can further comprise one or more cognitive drugs in an amount effective for enhancing cognitive function. The skilled artisan can determine the amount of cognitive drug to be added to the composition based upon the recommended dosage for the drug given by its manufacturer or upon the animal's weight, species, age, health status, and the like.

In certain embodiments, the daily dose for the compositions ranges from about 5 mg/day to about 5,000 mg/day, 10,000 mg/day, or 20,000, or more per animal. In one aspect, the daily dose ranges from about 30 mg/day to about 10,000 mg/day per animal, and in one specific aspect, from about 750 mg/day to about 7,500 mg/day per animal. The daily dose of LCPUFA, NORC, and MCTs can be measured in terms of grams of LCPUFA, NORC, and MCTs per kg of body weight (BW) of the animal. The daily dose of LCPUFA, NORC, and MCTs thereof can range from about 0.001 g/kg to about 50 g/kg BW of the animal, although greater or lesser doses can be provided. In one aspect, the daily dose of LCPUFA, NORC, and MCTs can be from about 0.001 g/kg to about 25 g/kg BW of the animal. In another aspect, the daily dose of LCPUFA, NORC, and MCTs thereof can be from about 0.001 g/kg to about 10 g/kg BW of the animal. In yet another aspect, the daily dose of LCPUFA, NORC, and MCTs can be from about 0.001 g/kg to about 5 g/kg BW of the animal. In still another aspect, the daily dose of LCPUFA, NORC, and MCTs can be from about 0.001 g/kg to about 1 g/kg BW of the animal. In another aspect, the daily dose of the LCPUFA, NORC, and MCTs can be from about 0.001 g/kg to about 0.5 g/kg BW of the animal.

Administration in accordance with the methods can be on an as-needed or as-desired basis of varying or regular frequency. A goal of regular ingestion is to provide the animal with a regular and consistent dose of the composition or the direct or indirect metabolites that result from such ingestion. Such regular and consistent dosing will tend to create constant blood levels of the components of the compositions or their direct or indirect metabolites. Thus, regular administration can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal, e.g., orally, or otherwise. The compositions can alternatively be contacted with, or admixed with, daily feed or food, including a fluid, such as drinking water, or an intravenous connection for an animal that is receiving such treatment. When utilized as a daily feed or food, administration will be well known to those of ordinary skill.

Administration can also be carried out as part of a dietary regimen for the animal. For example, a dietary regimen may comprise causing the regular ingestion by the animal of a composition described herein in an amount effective to prevent, reduce, or delay a decline in one or more motor or behavioral functions in the animal.

According to the methods of the invention, administration of the compositions, including administration as part of a dietary regimen, can span a period of time ranging from parturition through the adult life of the animal. In various embodiments, the animal can be a human or companion animal such as a dog or cat. In certain embodiments, the animal can be a young or growing animal. In more specific embodiments, the animal can be an aging animal. In some embodiments administration begins, for example, on a regular or extended regular basis, when the animal has reached more than about 30%, 40%, or 50% of its projected or anticipated lifespan. In some other embodiments, the animal has attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

In a further aspect, the invention provides kits suitable for administering a composition comprising one or more MCTs to an animal. The kits can comprise in separate containers in a single package or in separate containers in a virtual package, as appropriate for the kit component one or more MCTs and, optionally, one or more of (1) one or more other ingredients suitable for consumption by an animal; (2) one or more if LCPUFAs; (3) one or more of NORCs; (4) one or more B vitamins; (5) one or more antioxidants; (6) one or more cognitive drugs; (7) one or more prebiotics; (8) one or more probiotics; (9) one or more diagnostic devices suitable for determining whether an animal could benefit from compositions and methods for increasing mobility and/or activity; (10) instructions for how to combine or prepare the MCTs and any other ingredients provided in the kit for administration to an animal; (11) instructions for how to use the combined kit components, prepared kit components, or other kit components for the benefit of an animal; and (12) a device for administering the combined or prepared kit components to an animal. The components can be each provided in separate containers in a single package or in mixtures of various components in different packages. The kits may comprise the ingredients in various combinations. For example, the kit could comprise a mixture of one or more B vitamins and one or more antioxidants in one container and one or more other ingredients in one or more other containers. Similarly, the kit could comprise a mixture of LCPUFA and MCTs in one container and one or more other ingredients in one or more other containers. Other such combinations can be produced by the skilled artisan based upon the characteristics of the ingredients and their physical and chemical properties and compatibilities.

In another aspect, the invention provides a means for communicating information about or instructions for one or more of (1) using compositions of the present invention for increasing mobility and/or activity; (2) admixing the MCTs, or other components of the invention to produce a composition suitable for increasing mobility and/or activity; (3) using the kits of the present invention for increasing mobility and/or activity; and (4) administering the compositions to an animal. The means comprises one or more of a physical or electronic document, digital storage media, optical storage media, audio presentation, audiovisual display, or visual display containing the information or instructions. In one embodiment, the means can be selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising MCTs, and one or more other ingredients suitable for consumption by an animal, e.g., protein, fat, carbohydrate, fiber, B vitamins, and antioxidants. The methods comprise admixing one or more ingredients suitable for consumption by an animal with MCTs, and possibly other ingredients such as LCPUFA, NORC, B vitamins and/or antioxidants. Alternatively, the methods comprise applying MCTs, and other ingredients if desired, separately or in any combination onto the food composition, e.g., as a coating or topping. The MCTs can be added at any time during the manufacture and/or processing of the food composition. This includes, for example, admixing the MCTs as part of the core formulation of the “body” of the food composition or applying them as a coating, i.e., primarily to the surface of the food composition after its manufacture. The compositions can be made according to any method suitable in the art.

In another aspect, the present invention provides a package comprising a composition of the present invention and a label affixed to the package containing a word or words, picture, design, acronym, slogan, phrase, or other device, or combination thereof, that indicates that the contents of the package contains a composition suitable for increasing mobility and/or activity in an animal, e.g. an aging animal. Typically, such device comprises the words “improves activity”, “improves mobility”, “reduces inactivity”, or an equivalent expression printed on the package. Any package or packaging material suitable for containing the composition can be useful in the invention, e.g., a bag, box, bottle, can, pouch, and the like manufactured from paper, plastic, foil, metal, and the like. In one embodiment, the package contains a food composition adapted for a particular animal such as a human, canine or feline, as appropriate for the label, and in one aspect, a companion animal food composition.

### EXAMPLES

The invention can be further illustrated by the following example, although it will be understood that this example is included merely for purposes of illustration and is not intended to limit the scope of the invention unless otherwise specifically indicated.

### Example 1

### MCT Compositions for Increased Activity for Canines

Nine Senior Beagle dogs with ages between 7.9 to 11.3 (mean age=9.77 years old) were used. Each dog's maintenance energy requirement (MER) was determined by its food intake (A Super-premium-like control without MCT) that sustained its body weight. The dogs were fed 100% of their baseline MERs during the study. The dogs were fed the control diet for 5 days and their daily activity was recorded with commercial activity monitor. Then, the dogs were fed the Diet with 5.5% MCTs for 4 weeks with their daily activity recorded with commercial activity monitor. The activity was calculated as day activity (from 6 AM to 6 PM) and night activity (after 6 PM and before 6 AM). Table 1 outlines the nutrient composition of the control and MCT diets.

TABLE 1

| | Control | MCT Diet |
|---|---|---|
| Nutrient | (wt %) | (wt %) |
| Moisture | 12.00 | 8.50 |
| Crude Protein | 26.00 | 29.17 |
| Crude Fat | 16.00 | 13.90* |
| Crude Fiber | 3.00 | 2.96 |

*included 5.5 wt % MCT

Baseline and average weekly activities in senior dogs after MCT diet feeding are summarized in Table 2. The senior dogs fed the MCT diet showed increased both day and night activity during the 1st week of the MCT diet feeding, and higher activity at both day and night was maintained at the end of the 4-week feeding study. These data indicate that MCTs are able to increase mobility and activity and improve or treat frailty.

TABLE 2

| | Day Activity | Night Activity |
|---|---|---|
| Week | (counts/minute) | (counts/minute) |
| 0 | 92.28 | 33.86 |
| (Baseline) | | |
| 1 | 134.52 | 51.42 |
| 2 | 163.26 | 48.76 |
| 3 | 143.00 | 47.76 |
| 4 | 116.04 | 42.14 |

### Example 2

### MCT Compositions for Increased Activity for Felines

Pre-baseline maintenance energy requirement was determined for sixteen healthy cats between 7 and 12 years old. The cats' body condition score (BCS) was between 4 and 7. All cats were fed the control diet (protein: 41.7%; fat: 20.5%; moisture: 6.91%; fiber: 0.48%). Maintenance Energy Requirement (MER) was estimated for each cat during the pre-baseline phase. The equation for MER calculations was as follows: MER (kcal/day)=60×Body Weight (kg).

Baseline activity was determined for the cats as follows. The cats were fed the control diet (protein: 41.7%; fat: 20.5%; moisture: 6.91%; fiber: 0.48%) for one week with 100% of the estimated MERs and daily activity was recorded with activity monitor at every 15 s for one week.

All the cats were fed the MCT diet (protein: 42.48%; fat: 20.8%; moisture: 6.75%; fiber: 0.24%) with 100% of their baseline MERs for 4 weeks, and their daily activity was recorded with activity monitors at every 15 s for 4 weeks. The activity data were downloaded weekly.

Baseline and average weekly activities in cats after MCT diet feeding are summarized in Table 3. The cats fed the MCT diet showed increased both day and night activity during the 1st week of the MCT diet feeding, and higher activity at both day and night was maintained at the end of the 4-week feeding study. These data indicate that MCTs are able to increase mobility and activity and improve or treat frailty.

TABLE 3

| | Day Activity | Night Activity |
|---|---|---|
| | (total counts/ | (total counts/ |
| Week | 12 hours) | 12 hours) |
| 0 | 120692 | 30635 |
| (Baseline) | | |
| 1 | 151622 | 35767 |
| 2 | 156359 | 37845 |
| 3 | 153636 | 41853 |
| 4 | 167704 | 49113 |

In the specification, there have been disclosed typical embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for increasing mobility or activity in an animal or treating frailty in an animal, comprising:
identifying an animal with decreased mobility or activity, or having frailty; and
administering a composition comprising one or more medium chain triglycerides (MCTs) to the animal in a therapeutically amount effective for increasing mobility or activity in the animal or treating frailty in the animal.

2. The method of claim 1, wherein the animal is a companion animal.

3. The method of claim 1, wherein the animal is a cat or dog.

4. The method of claim 1, wherein the animal is an aging animal.

5. The method of claim 1, wherein the MCTs have the structure of Formula I: 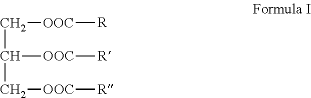
wherein the R, R′ and R″ esterified to the glycerol backbone are each independent fatty acids having 6-12 carbons.

6. The method of claim 5, wherein greater than about 95% of the R, R′ and R″ are 8 carbons in length.

7. The method of claim 6, wherein the remaining R, R′ and R″ are 6-carbon or 10-carbon fatty acids.

8. The method of claim 1, wherein the MCTs are administered as part of a pet food composition.

9. The method of claim 1, wherein the MCTs are administered in an amount of 0.001 g/kg to about 50 g/kg body weight of the animal.

10. The method of claim 1, wherein the MCTs are administered to the animal by adding the MCTs to an existing pet food composition prior to feeding the existing pet food composition to the animal.

11. A food composition suitable for increasing mobility or activity in an animal or treating frailty in an animal, comprising:
about 15% to about 50% protein;
about 5% to about 40% fat;
about 5% to about 50% carbohydrate; and
about 1% to about 40% one or more medium chain triglycerides (MCTs);
wherein the composition has a moisture content of about 5% to about 20% and the MCTs are present in a therapeutically amount effective for increasing mobility or activity or treating frailty in the animal.

12. The food composition of claim 11, wherein the MCTs are present in an amount from about 5% to about 30% and wherein the MCTs have the structure of Formula I: 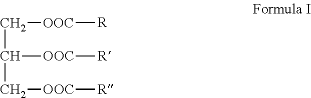
wherein the R, R′ and R″ esterified to the glycerol backbone are each independent fatty acids having 6-12 carbons.

13. The food composition of claim 12, wherein greater than about 95% of the R, R′ and R″ are 8 carbons in length.

14. The food composition of claim 13, wherein the remaining R, R′ and R″ are 6-carbon or 10-carbon fatty acids.

15. The food composition of claim 11, further comprising one or more B vitamins in an amount of about 0.1 to 40 times the recommended daily requirement of B vitamins.

16. The food composition of claim 15, wherein the one or more B vitamins include at least one of B1 (thiamine), B2 (riboflavin), B3 (aka P or PP) (niacin, including nicotinic acid and/or nicotinamide), B5 (pantothenic acid), B6 (pyridoxine), B7 (aka H) (biotin), B8 (myo-inositol), B9 (aka M or B-c) (folic acid), B12 (cobalamin), salts thereof, conjugates thereof, or derivatives thereof that are recognized or found to have B vitamin activity.

17. The food composition of claim 11, further comprising one or more antioxidants in an amount from about 0.01 to 25 times the recommended daily requirement of antioxidants.

18. The food composition of claim 17, wherein the one or more antioxidants include at least one of vitamin A, vitamin C, vitamin E, α-lipoic acid, chlorophyll, glutathione, ubiquinols, coenzyme Q10, carotenoids, lycopene, flavonoids, phenolic acids, polyphenols, pycnogenol, cherry, blackberry, strawberry, raspberry, crowberry, blueberry, bilberry/wild blueberry, black currant, pomegranate, grape, orange, plum, pineapple, kiwi fruit, grapefruit, kale, chili pepper, red cabbage, peppers, parsley, artichoke, Brussels sprouts, spinach, lemon, ginger, garlic, red beets, apricots, prunes, dates, broad beans, pinto beans, soybeans, pecans, walnuts, hazelnuts, ground nut, sunflower seeds, barley, millet, oats, corn, cloves, cinnamon, rosemary, oregano, Ginkgo biloba, uyaku, carica papaya, green tea, red wine, selenium, butylated hydroxyanisole (BHA) and butylated hydroxytoluene (BHT).

19. The food composition of claim 11, wherein the food composition is formulated as a complete and nutritionally balanced pet food.

20. The food composition of claim 11, wherein the food composition is formulated as a dry extruded pet food, as a treat for a pet, or as a semi-moisture pet food composition.
